(19) United States
(12) Patent Application Publication
(10) Pub. No.: US 2011/0065788 A1
(43) Pub. Date: Mar. 17, 2011

(54) AQUEOUS FORMULATIONS OF (2-HYDROXYMETHYL-INDANYL-4-OXY)-PHENYL-4,4,4-TRIFLUOROBUTANE-1-SULFONATE

(75) Inventors: Bernd Kühn, Frankfurt (DE); Antje Brück, Konstanz (DE); Yoshifumi Katakawa, Fujieda-Shi (JP); Masami Yasui, Koka-Gun (JP)

(73) Assignee: BAYER SCHERING PHARMA AKTIENGESELLSCHAFT, Berlin (DE)

(21) Appl. No.: 12/949,773

(22) Filed: Nov. 18, 2010

Related U.S. Application Data
(63) Continuation of application No. 10/510,908, filed on Aug. 1, 2005, now abandoned.

(30) Foreign Application Priority Data
Jul. 11, 2002 (DE) 10215942.4

(51) Int. Cl. A61K 31/255

(52) U.S. Cl. 514/510

(57) ABSTRACT

The invention relates to aqueous formulations containing (−)-(R)-3-(2-hydroxymethylindanyl-4-oxy)phenyl 4,4,4-trifluorobutane-1-sulfonate. Said formulations are suitable as infusion solutions or as concentrate for producing these infusion solutions.

The present invention relates to (−)-(R)-3-(2-hydroxymethylindanyl-4-oxy)phenyl 4,4,4-trifluorobutane-1-sulfonate-containing aqueous formulations which are suitable as infusion solutions or as concentrate for preparing such infusion solutions.

(−)-(R)-3-(2-Hydroxymethylindanyl-4-oxy)phenyl 4,4,4-trifluorobutane-1-sulfonate is a compound of the formula

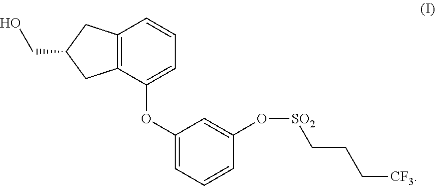

As a cannabinoid receptor agonist, the compound (I) is suitable for the prevention and treatment of stroke and craniocerebral trauma; it was described for the first time in example 278 of WO 98/37061. Aqueous pharmaceutical preparations suitable for parenteral administration are, however, not disclosed in WO 98/37061. Since it is advantageous in the acute treatment of stroke and craniocerebral trauma to administer the medicament as infusion solution, there was a need for aqueous formulations containing the compound (I) and appropriate for this purpose of use.

Remarkably, aqueous formulations of the compound (I) show an inhomogeneous concentration distribution. This means that, especially at low active ingredient concentrations of a few milligrams per liter, it must be assumed that an infusion rate which is constant based on the dose cannot be ensured over the complete infusion time. The disadvantages associated therewith are obvious.

For single-dose pharmaceutical forms, including parenteral powders and suspensions for injection, the pharmacopeias (Ph. Eur. 4, 2002) require testing for uniformity of content, a deviation which is as low as possible and does not exceed ±15% from the average of the active ingredient content.

It has surprisingly been found that the addition of cyclodextrin to aqueous formulations led to uniform concentration.

The invention thus relates to aqueous formulations comprising compound (I) and cyclodextrin.

Cyclodextrins and methods for preparing them are disclosed in U.S. Pat. No. 3,453,259, U.S. Pat. No. 3,459,731, WO 97/39770, U.S. Pat. No. 5,670,530, WO 96/32135, EP-B 149 197 and U.S. Pat. No. 4,727,064. Cyclodextrins are cyclic oligosaccharides which are formed in the degradation of starch by cyclodextrin glycosyl transferases.

β-Cyclodextrins contain seven α-1,4-linked glucose units. The 21 hydroxy groups present in this molecule can be wholly or partly substituted for example with optionally substituted aliphatic C₂-C₆ groups, preferably with hydroxypropyl or sulfobutyl groups. The cyclodextrins used in this case preferably have an average degree of substitution (DS) per molecule of from 1 to 10, in particular from 3 to 8.

The term “cyclodextrin” for the purposes of the invention includes the unsubstituted, the partially and the completely substituted cyclodextrins, especially hydroxypropyl- and sulfobutyl-substituted β-cyclodextrins.

Surprisingly, it additionally emerges that physiologically tolerated acids are able to increase the storage stability of the aqueous formulations.

Examples of such physiologically tolerated acids include mineral acids such as, for example, hydrochloric acid, sulfuric acid, mono- to 4-basic saturated and unsaturated C₂-C₁₀-carboxylic acids such as, for example, acetic acid, succinic acid, maleic acid, fumaric acid, C₂-C₆-hydroxy carboxylic acids such as, for example, malic acid, citric acid, glycolic acid, lactic acid, tartaric acid, cinnamic acid, C₃-C₆-keto carboxylic acids such as, for example, pyruvic acid, mono- or dibasic C₂-C₁₀-amino acids such as, for example, alanine, aspartic acid, glutamic acid, glycine, isoleucine, leucine, lysine, methionine, phenylalanine, proline, serine, threonine, valine, C₆-C₁₂-amido carboxylic acids such as, for example, hippuric acid, C₄-C₁₀-lactones such as, for example, ascorbic acid, and mixtures thereof. Lactic acid and citric acid are preferred; citric acid is particularly preferred.

A preferred pH range for the aqueous formulations of the invention is from 2 to 6, in particular 3 to 5 and specifically about 3.5 to 4.5.

To prepare an isotonic solution, the formulations of the invention can comprise compounds suitable for this purpose, such as, for example, glucose, mannitol, preferably sodium chloride. A solution is referred to as isotonic when it has an osmotic pressure of from 250 to 500, preferably 270 to 350, mosmol/kg.

Preferred isotonic formulations of the invention comprise from 5 to 15, preferably 7 to 13 and particularly preferably 8 to 10 g/l sodium chloride, based on the formulation ready for use.

It is additionally possible to add to the formulations of the invention physiologically tolerated organic solvents, for example polyethylene glycols, propylene glycol, glycofurol, glycerol or—preferably—alcohols, especially ethanol.

The formulations of the invention may generally comprise from 0.05 to 2, preferably 0.1 to 1.5 and in particular about 0.6 to 1.0 g/l organic solvent based on the formulation ready for use.

The formulations of the invention may be in the form of infusion solutions ready for use or of aqueous concentrates from which the infusion solutions can then be prepared by adding water or isotonic electrolyte solution. These concentrations of the invention may comprise the compound (I) in a concentration of from 0.002 to 9.0, preferably from 0.01 to 0.05, particularly preferably of 0.025 g/l. The concentrates may comprise cyclodextrin in concentrations of from 4 to 550, preferably from 20 to 200, particularly preferably of 50 g/l. A homogeneous solution can be prepared from the concentrates easily and quickly under sterile conditions and is suitable directly for use, for example as infusion solution.

The formulation of the invention may comprise cyclodextrin in 0.1 to 60, preferably 1 to 30, particularly preferably 1 to 10, in particular 2 g/l based on the formulation ready for use.

The solubility of the compound (I) in water is 0.002 g/l at 25° C.

The formulation of the invention ready for use for infusion may comprise an active ingredient concentration of from 0.00005 to 0.002, preferably 0.0001 to 0.002, in particular 0.0005 to 0.0015 and very specifically about 0.001 g of compound WA of solution.

The formulations of the invention can be prepared simply by mixing and dissolving the components.

It has generally proved advantageous to administer the compound (I) in total amounts of about 0.001 to about 240, preferably 0.01 to 24 μg/kg of body weight every 24 hours, where appropriate in the form of a plurality of single doses, to achieve the desired result.

It may, however, be advantageous where appropriate to deviate from the stated amounts, and in particular to do so as a function of the nature and body weight of the treated patient, of the individual behavior towards the medicament, the nature and severity of the disease, the mode of preparation and administration, and the time or interval over which administration takes place.

The invention further relates to an administration kit consisting of a container comprising the aqueous formulation and of an infusion apparatus. The infusion apparatus consists in the simplest case of a needle, connecting tubes, and a drip chamber. An infusion pump and regulating stopcocks can be attached to the connecting tubes. Administration is additionally possible by means of syringe drivers comprising infusion syringes with attached connecting tubes.

The materials of the administration kit which come into contact with the product can consist for example of polyethylene (PE), polypropylene (PP), polyamides, polyesters or copolymers thereof, acrylonitrile-butadiene-styrene copolymers, polypropylene/styrene-ethylene-butylene-styrene, preferably of polyolefins, particularly preferably of polyethylene.

### EXAMPLES

### 1) Example of an Infusion Formulation Ready for Use and Based on Hydroxypropyl-β-Cyclodextrin

Composition (in g/l)

| | |
|---|---|
| Compound (I) | 0.001 |
| Hydroxypropyl-β-cyclodextrin | 2 |
| (® Cavitron 82004, Cerestar) | |
| Sodium chloride | 9 |
| Ethanol for inj. | 0.8 |
| Citric acid | 0.016 |
| Water | 993.383 |

Preparation: a solution of compound (I) in ethanol is added with stirring to an aqueous solution of hydroxypropyl-(β-cyclodextrin and sodium chloride. The pH is adjusted to about 4 with citric acid. The solution is sterilized by filtration, dispensed into 250 ml glass bottles, closed with rubber stoppers and crimped caps and then sterilized in a steam autoclave at 121° C. for 20 min.

### 2) Example of an Infusion Formulation Ready for Use and Based on Sulfobutyl Ether β-Cyclodextrin

Composition (in g/l)

| | |
|---|---|
| Compound (I) | 0.001 |
| Sulfobutyl ether β-cyclodextrin | 2 |
| (® Captisol, CyDex) | |
| Sodium chloride | 9 |
| Ethanol for inj. | 0.8 |
| Citric acid | 0.016 |
| Water | 993.383 |

Preparation: a solution of compound (I) in ethanol is added with stirring to an aqueous solution of sulfobutyl ether (β-cyclodextrin and sodium chloride. The pH is adjusted to about 4 with citric acid. The solution is sterilized by filtration, dispensed into 250 ml glass bottles, closed with rubber stoppers and crimped caps and then sterilized in a steam autoclave at 121° C. for 20 min.

### 3) Example of a Concentrate for Preparing an Infusion Formulation

Composition (in g/l)

| | |
|---|---|
| Compound (I) | 0.025 |
| Hydroxypropyl-β-cyclodextrin | 50 |
| (® Cavitron 82004, Cerestar) | |
| Sodium chloride | 9 |
| Ethanol for inj. | 0.8 |
| Citric acid | 0.016 |
| Water | ad 1.01 |

Preparation: a solution of compound (I) in ethanol is added with stirring to an aqueous solution of hydroxypropyl-β-cyclodextrin and sodium chloride. The pH is adjusted to about 4 with citric acid. The solution is sterilized by filtration, dispensed into 10 ml glass bottles, closed with rubber stoppers and crimped caps and then sterilized in a steam autoclave at 121° C. for 20 min.

Before use, 10 mg of concentrate are mixed with 240 ml of physiological saline solution. The result is an infusion solution ready for use with an active ingredient concentration of 0.001 g/l.

What is claimed is:

1. An aqueous formulation comprising (−)-(R)-3-(2-hydroxymethylindanyl-4-oxy)phenyl 4,4,4-trifluorobutane-1-sulfonate (I) and cyclodextrin.

2. The formulation of claim 1, comprising from 0.00005 to 9.0 g/l of the compound (I) and from 0.1 to 550 g/l of cyclodextrin.

3. The formulation of claim 1, comprising from 0.0001 to 0.050 g/l of the compound (I) and from 0.2 to 200 g/l cyclodextrin.

4. The formulation of claim 1, comprising from 0.0005 to 0.025 g/l of the compound (I) and from 1 to 50 g/l cyclodextrin.

5. The formulation of claim 1, which has a pH of from 2 to 6.

6. The formulation of claim 1, comprising at least one physiologically tolerated acid.

7. The formulation of claim 6, which comprises citric acid as physiologically tolerated acid.

8. The formulation of claim 1, comprising from 8 to 10 g/l sodium chloride based on the formulation ready for use.

9. The formulation of claim 1, comprising from 0.05 to 2 g/l ethanol based on the formulation ready for use.

10. An administration kit consisting of a) a container comprising the aqueous formulation of claim 1, b) infusion apparatus, where at least the parts which come into contact with the product consist of polyethylene, polypropylene, polyester, polyamide, acrylonitrile-butadiene-styrene copolymers, polypropylene/styrene-ethylene-butylene-styrene or copolymers thereof.

11. The formulation of claim 1 comprising about 50 g/l of cyclodextrin.

12. The formulation of claim 1 comprising about 2 g/l of cyclodextrin.

13. The formulation of claim 1, wherein said formulation is suitable for parenteral administration.

14. An aqueous formulation comprising (−)-(R)-3-(2-hydroxymethylindanyl-4-oxy)phenyl 4,4,4-trifluorobutane-1-sulfonate (I) and from 1 to 50 g/l of cyclodextrin.

15. The formulation of claim 14, wherein the compound (I) is at a concentration of from 0005 to 0.025 g/l.

16. The formulation of claim 14, further comprising from 8 to 10 g/l sodium chloride based on the formulation ready for use.

17. The formulation of claim 14, further comprising from 0.05 to 2 g/l ethanol based on the formulation ready for use.

18. The formulation of claim 14, further comprising ethanol, sodium chloride, and citric acid.
